(19) United States
(12) Patent Application Publication
(10) Pub. No.: US 2024/0016519 A1
(43) Pub. Date: Jan. 18, 2024

(54) APPARATUS AND METHOD FOR REMOVING INTRA-OSSEOUS DEVICE

(71) Applicant: Shippert Tech, LLC, Boalsburg, PA (US)

(72) Inventor: Brian Shippert, Boalsburg, PA (US)

(21) Appl. No.: 17/863,115

(22) Filed: Jul. 12, 2022

(51) Int. Cl. A61B 17/34

(52) U.S. Cl. CPC A61B 17/3496; A61B 17/3472

(57) ABSTRACT

Apparatuses, systems, and methods are provided for securing an intra-osseous device within a safety device to prevent stick injuries and to guide the removal of the intra-osseous device from the bone of a patient. A body of the safety device has a cavity that receives the intra-osseous device, and the cavity guides the removal of the intra-osseous device along a straight line to prevent injury during removal. The intra-osseous device is drawn past a lock feature that secures the intra-osseous device within the body such that a needle of the intra-osseous device is contained within a cavity of the body and cannot injure the patient, staff members, etc.

### FIELD

The present disclosure provides an apparatus, system, and method for safely removing an intra-osseous device from a body into an enclosed containment unit.

### BACKGROUND

Every year, hundreds of thousands of sharps related injuries occur among healthcare workers. For example, according to the Center for Disease Control and Prevention (CDC) over 385,000 sharps related injuries occur annually. Aside from the stress and risk of blood borne pathogens associated with sharps injuries (Hepatitis B, Hepatitis C, Human Immunodeficiency Virus, syphilis, malaria, herpes, etc.), there are healthcare costs: loss of time, testing and treatment also increase costs and place the burden on healthcare workers, employers, and society.

Sharps safety is such a serious issue that the United States Congress passed the Needlestick Safety and Prevention Act and guides regulatory agencies like the Occupational Safety & Health Administration (OSHA) with regard to blood borne pathogen standards and mandates implementation of safer medical devices.

According to the National Institute of Occupational Safety and Health (NIOSH), the design of the device can increase the risk of injury. Specific features make certain devices more dangerous. These include: (NIOSH, 1999) devices with hollow-bore needles, needle devices that need to be taken apart or manipulated by the health care worker such as blood-drawing devices that need to be detached after use, syringes that retain an exposed needle after use, and needles that can be difficult to place in sharps containers.

Safety devices such as needleless or protected-needle systems, needle shielding, and encased needles have been effective at reducing accidental sharp exposures. However, there is a paucity of safety devices for medical apparatuses that gain access to bone marrow. Currently, medical devices that gain access to bone marrow, especially those that insert a penetrator or needle, contain all the specific features of a medical device at high risk for iatrogenic injuries.

Commonly known as intra-osseous devices, these devices present unique characteristics from other medical devices associated with sharps injuries. Since intra-osseous devices must enter cortical bone, they tend to be extremely sharp and typically have more than a single cutting surface or point to increase cortical bone penetration. Intra-osseous devices are typically of a larger caliber than commonly used needles and peripheral vascular access devices. Intra-osseous devices carry greater risk of bodily contamination due to microscopic fractured bone fragments in addition to blood component exposure.

The technique of removal of an intra-osseous medical device is a crucial step for patient and healthcare worker safety. Due to the bone matrix and micro-fractures associated with placement of the medical devices, withdrawal of these devices must occur in the opposite vector of the placement to minimize worsening of the fracture area. Angulation of the device during withdrawal is associated with increased chance of worsening micro-fractures of the bone and increased risk of device failure and lodgment in the subject bone.

The technique of removal can be complicated by initial incorrect placement of an intra-osseous device. Placement in dense cortical bone may necessitate significant force to overcome the initial resistance of the cortical bone. However, once initial resistance is overcome, the force exponentially decreases over a very short interval. The rapid decline in resistance creates another dangerous opportunity for sharps injury; healthcare workers have been known to physically jerk upon the device's release from the bone and potentially impale the worker or assistants during device removal.

### SUMMARY

In accordance with the present disclosure, an apparatus, system, and method for consistent and safe removal of an intra-osseous device into an enclosed locking unit are provided. In some embodiments, a safety device is configured to guide the removal of the intra-osseous device from a patient's body along a straight line and/or along the line that the intra-osseous device was inserted into the patient's body. As a result, the intra-osseous device does not damage bone or tissue as the intra-osseous device is removed.

In addition, as the intra-osseous device is removed, the safety device safely houses the intra-osseous device to prevent sharps related injuries to the patient, staff, etc. The intra-osseous device can generally have an aperture through which the intra-osseous device is drawn. The intra-osseous device passes a lock feature on the inner surface of the safety device, and the intra-osseous device is secured between the lock feature and a second end or aperture of the safety device. Once secured, the needle or other similar feature of the intra-osseous device is contained within the safety device to prevent sharps related injuries.

In accordance with one embodiment of the present disclosure, the body of the safety device has a frustum shape. The apex of the frustum has a central aperture with a diameter large enough to accommodate an average 12 mL or smaller syringe. The overall height of the frustum is greater than the length of the intra-osseous device. This allows the intra-osseous device to be completely enclosed in the safety device without the intra-osseous device's trochar or needle protruding outside of the containment of the safety device. Typically the overall height is 1.1-1.2 times the overall length of the intra-osseous device to be removed. An internal spatial volume within said frustum is sufficient for containing and securing the entirety of the intra-osseous device. The base of the frustum has a central aperture with a diameter that is between 1.1-1.2 times the maximum outer diameter of the intra-osseous device. The outer body of the frustum is solid to contain potential biologic material. The cavity within the safety device is cylindrical to maintain the correct vector for intra-osseous device removal. The device retaining and locking mechanism operates as a single unit. There are no intricate parts, no difficult manipulations, and no cumbersome features or structures to control. The safety device is structurally simple and easy to operate.

In accordance with another embodiment of the present disclosure, the body of the safety device has a frustum shape. The apex of the frustum has a central aperture in a diameter large enough to accommodate an average 12 mL or smaller syringe. The overall height of the frustum is greater than the length of the intra-osseous device. This allows the intra-osseous device to be completely enclosed in the device without the intra-osseous device's trochar or needle protruding outside of the containment of the apparatus. Typically, the overall height is 1.1-1.2 times the overall length of the intra-osseous device to be removed. An internal spatial volume within said frustum is sufficient for containing and securing the entirety of the intra-osseous device. The base of the frustum has a central aperture in a diameter 1.1-1.2 times the maximum outer diameter of the intra-osseous device to be removed.

Immediately proximal to the spatial volume required to contain the fully retracted intra-osseous device, is the integral locking mechanism, a ridge of material which constricts the internal circumference of the inner cylinder to a diameter that is smaller than the outer diameter of the intra-osseous device. Two narrow median grooves extend from the base of the frustum to a distance distal to the integral locking device. This distance is propriety to and dependent upon materials in order to allow natural flexing of the safety device to allow passage of the intra-osseous device beyond the locking ring into the fully retracted state. The cavity of the safety device is shaped as a cylinder. This design feature maintains the correct vector for intra-osseous device removal. The device retaining and locking mechanism operates as a single unit. There are no intricate parts, no difficult manipulations, and no cumbersome features or structures to control. The safety device is structurally simple and easy to operate.

In accordance with a further embodiment of the present disclosure, the body of the safety device has a cylindrical shape. One end of the cylinder has a central aperture in a diameter large enough to accommodate an average 12 mL or smaller syringe. The overall height of the cylinder is greater than the length of the intra-osseous device. This allows the intra-osseous device to be completely enclosed in the device without the intra-osseous device's trochar or needle protruding outside of the containment of the apparatus. Typically, the overall height is 1.1-1.2 times the overall length of the intra-osseous device to be removed. An internal spatial volume with said cylinder is sufficient for containing and securing the entirety of the intra-osseous device.

The other end of the cylinder has a central aperture in a diameter 1.1-1.2 times the maximum outer diameter of the intra-osseous device to be removed. The outer body of the cylinder is solid to contain potential biologic material. The cavity of the safety device is shaped as a cylinder. This design feature maintains the correct vector for intra-osseous device removal. The device retaining and locking mechanism operates as a single unit. There are no intricate parts, no difficult manipulations, and no cumbersome features or structures to control. The safety device is structurally simple and easy to operate.

In accordance with an embodiment of the present disclosure, the lock feature is positioned in the containment cylinder of the frustum immediately proximal to the fully retracted intra-osseous device. The locking mechanism is circumferential to allow several independent points of contact preventing disengagement of the intra-osseous device. The locking mechanism is a wedge with the widest portion most proximal to the fully retracted intra-osseous device. The narrowest portion of the wedge functions as the pivot point of the locking mechanism, facilitating movement of the locking mechanism out of the internal cylinder as the intra-osseous device is retracted fully into the containment cylinder. The wedge moves back into the lumen of the cylinder due to the structural integrity of the material the locking mechanism is composed of. With accordance with another embodiment of the present disclosure, the lock feature pushes the locking wedge back into the lumen of the containment cylinder.

In accordance with another embodiment of the present disclosure, the enclosure body of the safety device comprises two or more sections that clamp around an intra-osseous device such as a syringe. As described herein, the safety device helps prevent unintentional tissue damage and needle sticks by keeping the intra-osseous device in proper alignment and by safely housing the needle of an intra-osseous device after use. However, the initial joining of the safety device and the intra-osseous device can also present a possibility of unintentional needle sticks.

Specifically, the longitudinal movement of the intra-osseous device into the safety device combined with the relatively small cross-sectional areas of the intra-osseous device and safety device pose an increased possibility of an unintentional needle stick to a person handling the intra-osseous device and safety device.

With a safety device that has a clamping ability, the safety device can be joined to the intra-osseous device from a lateral direction and not a longitudinal direction, thus reducing the likelihood of an unintentional needle stick. In some embodiments, the enclosure body of the safety device can have a clamshell-like design with two sections that initially open to receive the intra-osseous device. Then, the sections close around the intra-osseous device to a closed position. Optionally, snap lock features can secure the two sections in the closed position. However, it will be appreciated that any lock feature can secure the two sections in the closed position.

In accordance with an embodiment of the present disclosure, deflectable guide tabs ensure a smooth retraction of the intra-osseous device from a patient, and then help hold the intra-osseous device in a secure position where the needle of the intra-osseous device is carefully positioned within the safety device. In various embodiments, a tab is positioned on an interior surface of each of two sections of the enclosure body, the tabs are oriented longitudinally, and lower ends of the tabs are located at the lower aperture of the safety device. As described elsewhere herein, the lower aperture of the safety device is large enough to allow any part of the intra-osseous device to pass through. As the intra-osseous device is retracted, the base of the intra-osseous device is channeled along the tabs, and the tab eventually form an inner diameter that is less than the outer diameter of the base. Thus, as the base continues to move through the safety device, the tabs deflect outward. Once the base clears the tabs, the tabs deflect inward and trap the base between the upper ends of the tabs and a lock feature to secure the intra-osseous device.

One particular embodiment of the present disclosure is an apparatus for securing an intra-osseous device, comprising a body extending from a first end to a second end; a cavity defined by an inner surface of the body, and the cavity extending between a first aperture at the first end of the body and a second aperture at the second end of the body; a lock feature positioned on the inner surface of the body, wherein the lock feature is positioned a first predetermined distance from the first end of the body; and a channel extending from the first end to a distal point that is a second predetermined distance from the first end, wherein the second predetermined distance is larger than the first predetermined distance.

In some embodiments, the lock feature comprises a ramp portion oriented towards the first end of the body and comprises a shoulder portion oriented towards the second end of the body, wherein the shoulder portion extends from the inner surface of the body at a substantially perpendicular angle. In various embodiments, the ramp portion extends from the inner surface of the body at an angle between approximately 30 and 60 degrees. In some embodiments, the lock feature extends continuously along the inner surface of the body between the channel and an additional channel.

In various embodiments, the lock feature is part of a plurality of discrete lock features positioned on the inner surface of the body. In some embodiments, the apparatus further comprises a relief aperture positioned at the distal point of the channel, wherein the relief aperture has an outermost width that is larger than a width of the channel. In various embodiments, the channel extends in a direction that is substantially parallel to a longitudinal axis of the body. In some embodiments, a cross-sectional profile of the lock feature has a semi-circular shape.

Another particular embodiments of the present disclosure is a system for securing an intra-osseous device, comprising a body extending from a first end to a second end; a cavity defined by an inner surface of the body, and the cavity extending between a first aperture at the first end of the body and a second aperture at the second end of the body; a lock feature positioned on the inner surface of the body, wherein the lock feature is positioned a predetermined distance from the first end of the body, and the lock feature defines an inner diameter; and an intra-osseous device having a base portion and a needle, wherein the base portion has an outermost diameter that is larger than the inner diameter defined by the lock feature, and wherein the intra-osseous device is configured to pass the lock feature such that the intra-osseous device is secured within the body and the needle is contained with the cavity.

In various embodiments, wherein the second aperture has an inner diameter that is smaller than the outermost diameter of the base portion such that the intra-osseous device is retained within the body between the second aperture and the lock feature after the intra-osseous device passes the lock feature. In some embodiments, the system further comprises a syringe selectively connected to the intra-osseous device, the syringe having a barrel with an outer diameter that is smaller than an inner diameter of the second aperture. In various embodiments, the inner diameter of the second aperture is between 1.1-1.2 times larger than the outer diameter of the barrel. In some embodiments, an overall length of the intra-osseous device is smaller than an overall length of the body. In various embodiments, a channel extends from the first end to a distal point that is a second predetermined distance from the first end, wherein the second predetermined distance is larger than the predetermined distance of the lock feature.

Yet another particular embodiment of the present disclosure is a method for securing an intra-osseous device in a safety device, comprising providing an intra-osseous device with an outermost diameter; providing a safety device having a body positioned about the intra-osseous device, wherein the body has an inner surface that defines a cavity extending between a first aperture at a first end of the body and a second aperture at a second end of the body, and wherein an inner diameter of the second aperture is smaller than the outermost diameter of the intra-osseous device; providing a lock feature on the inner surface of the body, wherein the lock feature defines an inner diameter that is smaller than the outermost diameter of the intra-osseous device; drawing the intra-osseous device through the body of the safety device towards the second end; and deflecting the lock feature with the intra-osseous device such that the intra-osseous device passes the lock feature and is contained with the cavity.

In some embodiments, the intra-osseous device has an overall length, wherein the overall length of the intra-osseous device is smaller than an overall length of the body of the safety device. In various embodiments, the method further comprises selectively connecting a syringe to the intra-osseous device, wherein the syringe has a barrel with an outer diameter that is less than the inner diameter of the second aperture; and removing the syringe from the intra-osseous device after the intra-osseous device is secured between the lock feature and the second aperture. In some embodiments, the method further comprises fastening the intra-osseous device to an osseous area. In various embodiments, the method further comprises providing a channel in the body of the safety device that extends from the first end to a distal point, wherein the channel allows the lock feature to deflect and permit the intra-osseous device to pass the lock feature. In some embodiments, the lock is positioned a first predetermined distance from the first end of the body, and the distal point of the channel is a second predetermined distance from the first end of the body, wherein the second predetermined distance is larger than the first predetermined distance.

One particular embodiment of the present disclosure is a safety device for an intra-osseous device, the safety device comprising an enclosure body having a first section and a second section, wherein the first and second sections are connected with a joint so that the first and second sections can move between an open position and a closed position, wherein the enclosure body holds the intra-osseous device in the closed position; an internal cavity defined by the enclosure body when the first and second sections are in the closed position, wherein the internal cavity extends between a first aperture and a second aperture, and a diameter of the first aperture is larger than a diameter of the second aperture; and a lock feature on interior surfaces of the first and second sections, wherein an inner diameter defined by the lock feature is less than the diameter of the first aperture to secure part of the intra-osseous device within the safety device.

In some embodiments, the safety device further comprises a first tab on the interior surface of the first section; a second tab on the interior surface of the second section, wherein the first and second tabs taper from the diameter of the first aperture to a smaller diameter at upper ends of the first and second tabs; and a gap between the upper ends of the first and second tabs and the lock feature, wherein part of the intra-osseous device is adapted to deflect the first and second tabs outward until the part passes the first and second tabs, then the first and second tabs deflect inward, and the part is secured within the gap. In various embodiments, the upper ends of the first and second tabs are closer to the second aperture at a second end of the enclosure body than the first aperture at a first end of the enclosure body. In some embodiments, the smaller diameter defined by the upper ends of the first and second tabs is less than the inner diameter defined by the lock feature. In various embodiments, the first tab is connected to the first section at only a lower end of the first tab, and the second tab is connected to the second section at only a lower end of the second tab.

In some embodiments, the safety device further comprises a protrusion extending from the first section; and a recess extending into the second section, wherein the protrusion extends into the recess to secure the first and second sections in the closed position. In various embodiments, the protrusion has a ramp portion and a shoulder portion, wherein the ramp portion contacts an edge of the second section to defect the protrusion as the protrusion extends into the recess, and the shoulder portion retains the protrusion within the recess and secures the first and second sections in the closed position.

In some embodiments, the safety device further comprises at least one support rib of the first and second sections that is positioned between the lock feature and the second aperture, wherein the at least one support rib defines an aperture with a diameter that is equal to the diameter of the second aperture. In various embodiments, the enclosure body has an exterior surface defined by the first and second sections, and the exterior surface tapers from a first diameter at a first end of the enclosure body to a smaller second diameter at a second end of the enclosure body. In some embodiments, the enclosure body is made from a material that allows at least some light to pass through the enclosure body.

Another particular embodiment of the present disclosure is a method of using a safety device with an intra-osseous device, comprising providing the safety device that has an enclosure body with a first section and a second section that are moveable between an open position and a closed position; a lock feature on interior surfaces of the first and second sections; positioning the intra-osseous device within the enclosure body and moving the first and second sections from the open position to the closed position to hold the intra-osseous device within the enclosure body; and moving the intra-osseous device through the enclosure body along a longitudinal axis of the enclosure body until the lock feature secures a base portion of the intra-osseous device, and a needle of the intra-osseous device is safety positioned within the enclosure body.

In some embodiments, the method further comprises deflecting a first tab on the first section and deflecting a second tab on the second section as the intra-osseous device is moved through an internal cavity of the enclosure body until the base portion passes upper ends of the first and second tabs, then the first and second tabs deflect back inward, and the base portion is secured between the upper ends of the first and second tabs and the lock feature. In various embodiments, the method further comprises inserting a protrusion of the first section into a recess of the second section to secure the first and second sections in the closed position.

In some embodiments, the method further comprises contacting a ramp portion of the protrusion against an edge of the second section as the protrusion is inserted into the recess to deflect the protrusion from an original position to a deflected position; and moving the ramp portion past the edge such that the protrusion reverts to the original position, and a shoulder of the protrusion retains the protrusion within the recess and secures the first and second sections in the closed position. In various embodiments, the method further comprises providing the safety device that has an internal cavity extending between a first aperture at a first end of the enclosure body and a second aperture at a second end of the enclosure body, wherein diameter of the first aperture is larger than a diameter of the second aperture such that the base portion can pass through the first aperture but not the second aperture. In some embodiments, the method further comprises removing the base portion of the intra-osseous device from a barrel of the intra-osseous device to leave the base portion and the needle of the intra-osseous device safely positioned within the enclosure body.

In various embodiments, the method further comprises extending the intra-osseous device into an osseous material as the intra-osseous is held within the safety device; and extending a plunger into a barrel of the intra-osseous device to deposit a fluid stored within the barrel to the osseous material. In some embodiments, the method further comprises extending the intra-osseous device into an osseous material as the intra-osseous is held within the safety device; and retracting a plunger from a barrel of the intra-osseous device to remove part of the osseous material. In various embodiments, the method further comprises guiding the intra-osseous device along the longitudinal axis with a first support rib extending from the first section and a second support rib extending from the second section, wherein the first and second support ribs define an inner diameter that is equal to an inner diameter defined by the lock feature.

A further particular embodiment of the present disclosure is a safety device for an intra-osseous device, the safety device comprising an enclosure body having a first section and a second section, wherein the first and second sections are connected with a joint so that the first and second sections can move between an open position and a closed position, wherein the enclosure body holds the intra-osseous device in the closed position; a protrusion extending from the first section; a recess extending into the second section, wherein the protrusion extends into the recess to secure the first and second sections in the closed position; an internal cavity defined by the enclosure body when the first and second sections are in the closed position, wherein the internal cavity extends between a first aperture and a second aperture, and a diameter of the first aperture is larger than a diameter of the second aperture; a lock feature on interior surfaces of the first and second sections, wherein an inner diameter defined by the lock feature is less than the diameter of the first aperture; a first tab on the interior surface of the first section; a second tab on the interior surface of the second section, wherein the first and second tabs taper from the diameter at the first aperture to a smaller diameter defined by upper ends of the first and second tabs; and a gap between the upper ends of the first and second tabs and the lock feature, wherein part of the intra-osseous device has a diameter that is larger than the diameter of the first aperture but less than the inner diameter defined by the lock feature such that the part of the intra-osseous device is adapted to deflect the first and second tabs outward until the part passes the first and second tabs, then the first and second tabs deflect inward, and the part is secured within the gap.

The Summary of the Invention is neither intended nor should it be construed as being representative of the full extent and scope of the present disclosure. The present disclosure is set forth in various levels of detail in the Summary of the Invention as well as in the attached drawings and the Detailed Description of the Invention and no limitation as to the scope of the present disclosure is intended by either the inclusion or non-inclusion of elements or components. Additional aspects of the present disclosure will become more readily apparent from the Detailed Description, particularly when taken together with the drawings. Further depictions of an embodiment of the present disclosure can be found in the Appendix, the entire disclosure of which is hereby incorporated herein by reference.

The above-described embodiments, objectives, and configurations are neither complete nor exhaustive. As will be appreciated, other embodiments of the disclosure are possible using, alone or in combination, one or more of the features set forth above or described in detail below.

The phrases “at least one,” “one or more,” and “and/or,” as used herein, are open-ended expressions that are both conjunctive and disjunctive in operation. For example, each of the expressions “at least one of A, B, and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together.

Unless otherwise indicated, all numbers expressing quantities, dimensions, conditions, and so forth used in the specification and claims are to be understood as being modified in all instances by the term “about.”

The term “a” or “an” entity, as used herein, refers to one or more of that entity. As such, the terms “a” (or “an”), “one or more,” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein.

It shall be understood that the term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials, or acts and the equivalents thereof shall include all those described in the summary of the invention, brief description of the drawings, detailed description, abstract, and claims themselves.

### BRIEF DESCRIPTION OF THE DRAWINGS

The accompanying drawings, which are incorporated in and constitute a part of the specification, illustrate embodiments of the disclosure and together with the Summary of the Invention given above and the Detailed Description of the drawings given below, serve to explain the principles of these embodiments. In certain instances, details that are not necessary for an understanding of the disclosure or that render other details difficult to perceive may have been omitted. It should be understood, of course, that the disclosure is not necessarily limited to the particular embodiments illustrated herein. Additionally, it should be understood that the drawings are not necessarily to scale.

FIG. 1A depicts a perspective view of a safety device for removing and securing an intra-osseous device in accordance with an embodiment of the present disclosure;

FIG. 1B is a cross-sectional view of the safety device of FIG. 1A taken along line B-B in accordance with an embodiment of the present disclosure;

FIG. 2 is a further perspective view of another safety device for removing and securing an intra-osseous device in accordance with an embodiment of the present disclosure;

FIG. 3A is a perspective view of a safety device positioned about an intra-osseous device in accordance with an embodiment of the present disclosure;

FIG. 3B is a perspective view of a safety device guiding an intra-osseous device during removal in accordance with an embodiment of the present disclosure;

FIG. 3C is a perspective view of a safety device securing an intra-osseous device during removal in accordance with an embodiment of the present disclosure;

FIG. 4A is a perspective view of another safety device for removing and securing an intra-osseous device in accordance with an embodiment of the present disclosure;

FIG. 4B is a cross-sectional view of the safety device of FIG. 4A taken along line B-B in accordance with an embodiment of the present disclosure;

FIG. 4C is another cross-sectional view of the safety device of FIG. 4A taken along line C-C in accordance with an embodiment of the present disclosure;

FIG. 5A is a perspective view of another safety device for removing and securing an intra-osseous device in accordance with an embodiment of the present disclosure;

FIG. 5B is a cross-sectional view of the safety device of FIG. 5A taken along line B-B in accordance with an embodiment of the present disclosure;

FIG. 5C is another cross-sectional view of the safety device of FIG. 5A taken along line C-C in accordance with an embodiment of the present disclosure;

FIG. 6A depicts a perspective view of a safety device for removing and securing an intra-osseous device in accordance with a further embodiment of the present disclosure;

FIG. 6B is a perspective view of the safety device in FIG. 6A with an intra-osseous device in accordance with a further embodiment of the present disclosure;

FIG. 6C is a perspective view of the safety device in FIG. 6A with an intra-osseous device in a first position in accordance with a further embodiment of the present disclosure;

FIG. 6D is a perspective view of the safety device in FIG. 6A with an intra-osseous device in a second position in accordance with a further embodiment of the present disclosure;

FIG. 7A is a cross-sectional view of the safety device and intra-osseous device in FIG. 6C taken along line C-C in accordance with a further embodiment of the present disclosure;

FIG. 7B is a cross-sectional view of the safety device and intra-osseous device in FIG. 6D taken along line D-D in accordance with a further embodiment of the present disclosure;

FIG. 8A is a front elevation view of the safety device in FIG. 6A in accordance with a further embodiment of the present disclosure; and

FIG. 8B is a side elevation view of the safety device in FIG. 6A in accordance with a further embodiment of the present disclosure.

Similar components and/or features may have the same reference label. Further, various components of the same type may be distinguished by following the reference label by a letter that distinguishes among the similar components. If only the first reference label is used, the description is applicable to any one of the similar components having the same first reference label irrespective of the second reference label.

### DETAILED DESCRIPTION

The present disclosure has significant benefits across a broad spectrum of endeavors. It is the Applicant's intent that this specification and the claims appended hereto be accorded a breadth in keeping with the scope and spirit of the disclosure being disclosed despite what might appear to be limiting language imposed by the requirements of referring to the specific examples disclosed. To acquaint persons skilled in the pertinent arts most closely related to the present disclosure, a preferred embodiment that illustrates the best mode now contemplated for putting the disclosure into practice is described herein by, and with reference to, the annexed drawings that form a part of the specification. The exemplary embodiment is described in detail without attempting to describe all of the various forms and modifications in which the disclosure might be embodied. As such, the embodiments described herein are illustrative, and as will become apparent to those skilled in the arts, may be modified in numerous ways within the scope and spirit of the disclosure.

Although the following text sets forth a detailed description of numerous different embodiments, it should be understood that the detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims. To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning.

Various embodiments of the present disclosure are described herein and as depicted in the drawings. It is expressly understood that although the figures depict safety devices, intra-osseous device, etc., the present disclosure is not limited to these embodiments

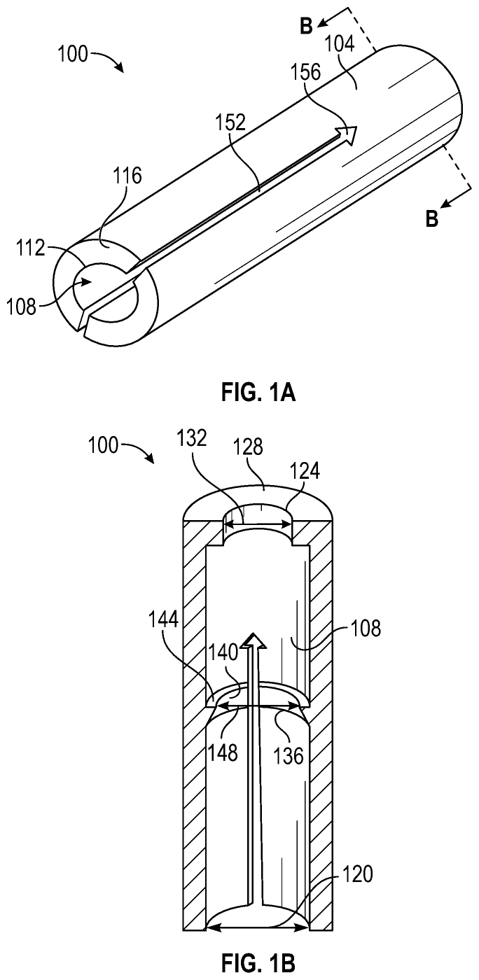

With reference to FIGS. 1A-B, a safety device 100 that includes an enclosure body 104 in accordance with embodiments of the present disclosure is depicted in a perspective view (FIG. 1A) and a cross-sectional view (FIG. 1B) taken along line B-B in FIG. 1A. The enclosure body 104 in this exemplary embodiment is in the form of a cylinder but could be other forms such as a frustum shape. A cavity 108 extends from a first aperture 112 in a bottom or first end 116 of the enclosure body 104 to a second aperture 124 in a top or second end 128 of the enclosure body 104. The first aperture 112 can have a diameter that is larger than the second aperture 124 to accommodate an intra-osseous device as described in further detail below. The cavity 108 defines a chamber with a substantially cylindrical volume, except for a retaining structure or lock feature 136 formed between the first 112 and second 124 apertures.

The lock feature 136 can be in the form of a wedge with a ramp portion 140 facing the first aperture 112 and a shoulder portion 144 facing the second aperture 124. The lock feature 136 can be formed continuously around the inner surface of the body 104, or can be formed as discrete ramp members, with gaps therebetween. In accordance with at least some embodiments of the present disclosure, the enclosure body 104 can include one or more channels 152 that extend from the first end 116 of the body 104 to a point between the lock feature 136 and the second end 128 of the body 104. The channels 152 can increase the flexibility of the body 104, and in particular can facilitate passing a base portion of an intra-osseous device past the lock feature 136 when withdrawing an intra-osseous device from a body, as discussed elsewhere herein. In accordance with at least some embodiments of the present disclosure, an end of each of the channels 152 opposite the first end 116 of the body 104 can have a stress relieving aperture or feature 156.

Also shown in FIG. 1B are an inner diameter 120 of the first aperture, an inner diameter 132 of the second aperture 124, and an inner diameter 148 defined by the lock feature 136. As described below these diameters 120, 132, 148 function with an outermost diameter of an intra-osseous device such that the outermost diameter can freely pass through the inner diameter 120 of the first aperture, pass through the inner diameter 148 of the lock feature 136 with force, and then be retained between the inner diameter 148 of the lock feature 136 and the inner diameter 132 of the second aperture 124.

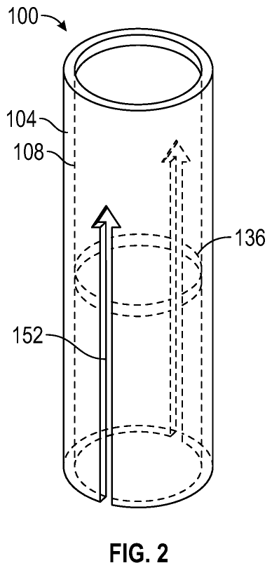

With reference to FIG. 2, a safety device 100 that includes an enclosure body 104 in accordance with other embodiments of the present disclosure is depicted in a transparent perspective view. The enclosure body 104 in this exemplary embodiment is in the form of a cylinder. Similar to other embodiments of a safety device 100 in accordance with embodiments of the present disclosure, a cavity 108 extends from a first aperture in a bottom or first end of the enclosure body 104 to a second aperture in a top or second end of the enclosure body 104. Also similar to other embodiments of the present disclosure, the cavity 108 can include a containment or primary chamber that defines a substantially cylindrical volume that extends from the first aperture to the second aperture, except for a retaining structure or lock feature 136 formed between the first and second apertures.

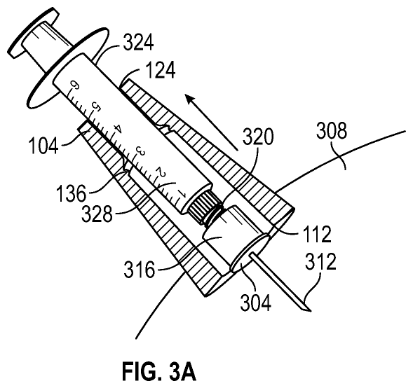

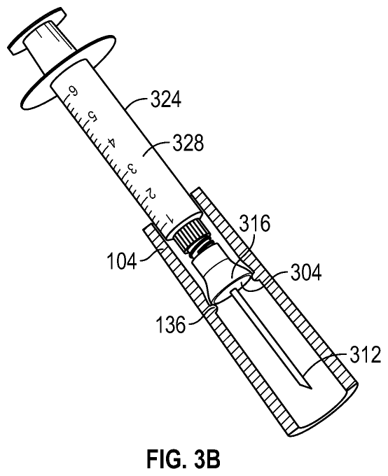

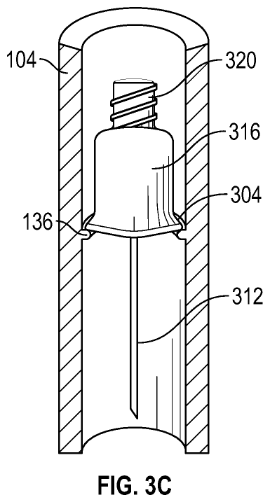

With reference now to FIGS. 3A-C, a safety device 100 in accordance with embodiments of the present disclosure is shown being used to safely guide and secure an intra-osseous device 304 in a body 104 of a safety device. As can be appreciated by one of skill in the art, the intra-osseous device 304 is typically placed in the patient body 308, and more particularly within an interior portion of a bone, using a drill (not shown) to rotate the intra-osseous device 304. The drill is then disengaged from the intra-osseous device 304. An IV or syringe 324 can then be connected to an attachment mechanism 320, such as a Luer lock, provided by a base portion 316 of the intra-osseous device 304, to enable fluids, such as medications, to be introduced to the patient body 308.

As can be appreciated by one of skill in the art, removing the intra-osseous device 304 from the body is preferably done by pulling the intra-osseous device 304 from the patient body 308 along a line that is coincident with the line along which the intra-osseous device 304 was installed. In addition, the tip of the intra-osseous device 304 is usually extremely sharp, and presents a stick hazard to personnel removing the intra-osseous device 304. A safety device 100 in accordance with embodiments of the present disclosure provides a stabilizing structure for removing the intra-osseous device 304, and further provides containment for the intra-osseous device 304, to avoid contact between the intra-osseous device 304 and users of the safety device 100.

In accordance with embodiments of the present disclosure, a process for removing the intra-osseous device 304 includes placing the first aperture 112 of the enclosure body 104 of the safety device 100 over the base portion 316 of the intra-osseous device 304. A syringe 324 or other removal tool can then be joined to the attachment mechanism 320. In order to facilitate applying a removing force that is coincident or nearly coincident with the line along which the device 304 is installed, the diameter of the first aperture 112 and the primary chamber can be dimensioned so that they are slightly (e.g. 0.5-2 mm) larger than the diameter of the base portion 316 of the intra-osseous device 304. In addition, the second aperture 124 can be dimensioned so that it has a diameter that is slightly (e.g. 0.5-2 mm) larger than a diameter of the syringe 324 barrel 328. The second aperture 124 can also extend along a line that is coincident with a central axis of the enclosure body 104 for some distance (e.g. 5-15 mm) to assist in stabilizing the syringe 324. A physician or other practitioner can then hold the enclosure body 104 against the patient body 308 with one hand, while pulling the syringe 324 barrel 328 away from the patient body 308. As or after the tip of the intra-osseous device 304 is completely withdrawn from the body, the base portion 316 of the intra-osseous device 304 is pulled past the lock feature 136. In accordance with embodiments of the present disclosure, pulling the base portion 316 of the intra-osseous device 304 past the lock feature 136 requires some force, and at least temporarily deforms at least one of the lock feature 136 or the base portion 316. The intra-osseous device 304 is then retained by the lock feature 136, and contained entirely within the interior chamber, for example as depicted in FIG. 3C.

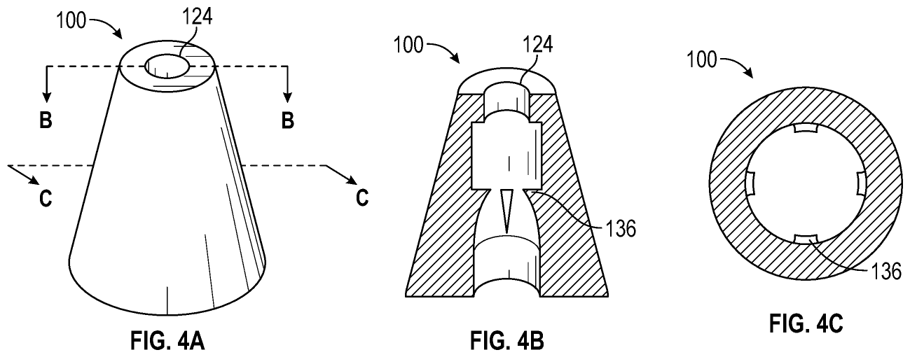

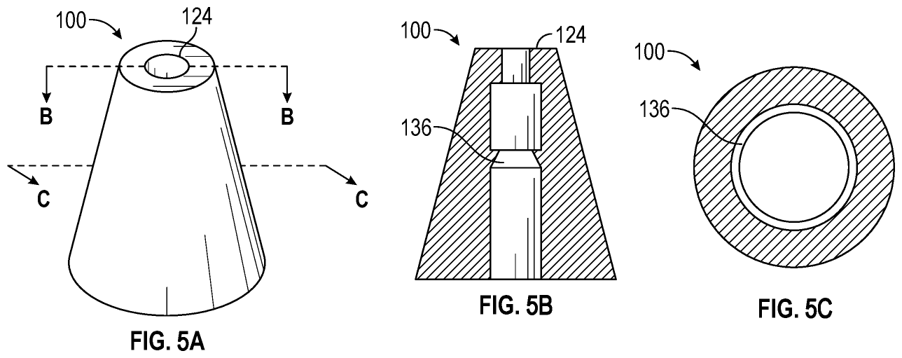

With reference to FIGS. 4A-4C and FIGS. 5A-5C, the safety device 100 may have a frustum shape. The lock feature 136 can be in the form of a wedge with a ramp portion facing the first aperture 112 a shoulder portion facing the second aperture 124. In accordance with some embodiments of the present disclosure, for example as depicted in FIGS. 4B and 4C, the lock feature 136 can be provided as one or more discrete ramp members or wedges, spaced around the inner diameter of the containment chamber. Although four discrete ramp members are show in FIGS. 4B and 4C, any number of ramp members can be included. Moreover, the portion of the inner surface of the body 104 occupied by the ramp members can be varied. Where the lock feature 136 is provided as more than one discrete ramp member, the different ramp members can be positioned at the same distance from the first aperture. In accordance with other some embodiments of the present disclosure, for example as depicted in FIGS. 5B-5C, the lock feature 136 can extend around the inner diameter of the containment chamber to form a continuous ring. In accordance with still other embodiments of the present disclosure, the lock feature 136 can include a spring or other elastic member, alone or in combination with a moveable tab. In at least some embodiments, the lock feature 136 is formed as an integral part of the enclosure body 104. In accordance with at least some embodiments of the present disclosure, the enclosure body 104 is formed from single, integral piece of material. The material used to form the body 104 can be, for example, a nylon, plastic, polycarbonate, or other material.

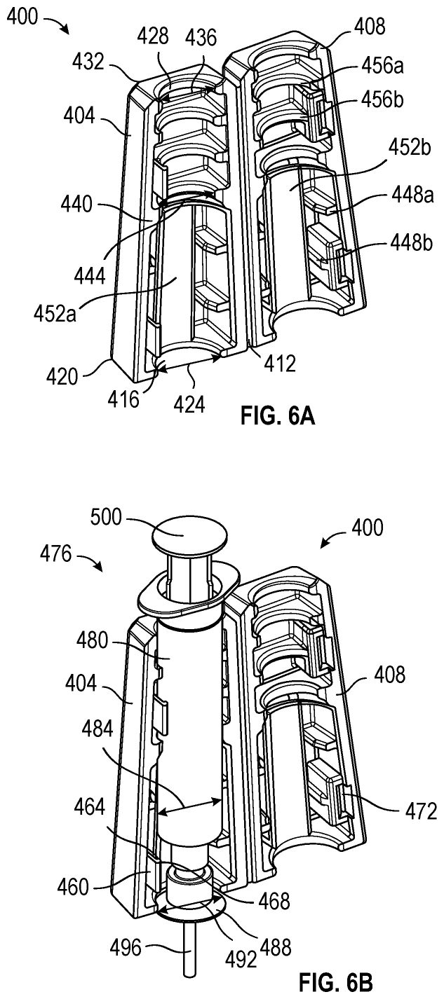

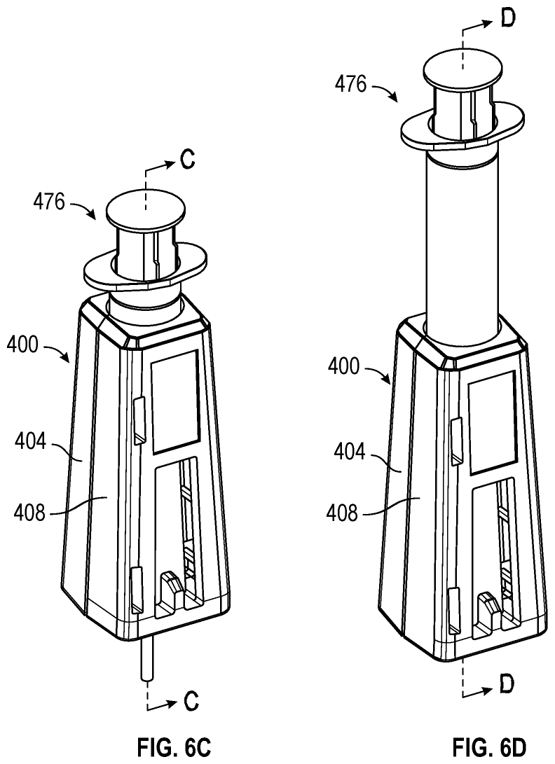

With reference to FIGS. 6A and 6B, a further embodiment of the safety device 400 with an enclosure body comprised of multiple sections is provided. Specifically, the safety device 400 has a first section 404 and a second section 408 connected at a joint 412. The first and section sections 404, 408 can move about this joint 412 between an open position as shown in FIGS. 6A-6B and a closed position shown in, for instance, FIGS. 6C and 6D. The joint 412 in this embodiment operates like a hinge. Thus, the joint 412 can be a thin, flexible structure that is molded along with the first and second sections 404, 408 as a single component. As the first and second sections 404, 408 move from an open position to a closed position, the joint 412 flexes to accommodate the movement. In further embodiments, the joint 412 can be a first hinge portion on the first section 404 and a second hinge portion on the second section 408. Each hinge portion has at least one knuckle, and a pin extends through the knuckles of each hinge portion to form the complete hinge. It will be appreciated that other hinge or hinge-like structures can accommodate the movement of the first and second sections 404, 408 between the open and closed positions.

Moreover, the joint 412 in the depicted embodiment has a single degree of freedom. However, it will be appreciated that other embodiments of the safety device 400 can include other types of joints with more degrees of freedom. In yet further embodiments, the safety device 400 has no joint 412, and the sections 404, 408 are completely separate in the open position, and then the sections 404, 408 are joined by, for example, snap lock features in the closed position. Similarly, other embodiments of the safety device 400 can have more than two sections 404, 408.

Next, like other embodiments described herein, the safety device 400 has a first aperture 416 at a first end 420 of the enclosure body and a second aperture 428 at a second end 432 of the enclosure body. Moreover, a lock feature 440 is positioned between the first and second apertures 416, 428. In contrast to other embodiments described herein, these features 416, 428, 440 are defined by multiple sections 404, 408 rather than a single enclosure body. As shown in FIG. 6A, the apertures 416, 428 are defined by a continuous edge once the sections 404, 408 are in the closed position. Similarly, the lock feature 440 forms a continuous edge once the sections 404, 408 are in the closed position. It will be appreciated that one or more or all of these features 416, 428, 440 can form, or be formed by, a segmented or otherwise non-continuous edge or structure once the sections 404, 408 are in the closed position.

In the depicted embodiment, the diameter 424 of the first aperture 416 is larger than the inner diameter 444 of the lock feature 440 and larger than the diameter 436 of the second aperture 428. As a result, some parts of the intra-osseous device pass through all apertures 416, 428 and the lock feature 440 whereas at least one part of the intra-osseous device passes through the first aperture 416 but not the lock feature 440. As a result, the safety device 400 fulfills the functions of retracting the intra-osseous device from a patient in a safe, linear manner, and then safely storing a needle of the intra-osseous device within the safety device 400.

FIGS. 6A and 6B also depict several ribs 448a, 448b, 456a, 456b that serve several functions. In this embodiment, the safety device 400 has two lower ribs 448a, 448b of the first section 404 and two corresponding lower ribs of the second section 408. These lower ribs 448a, 448b add rigidity to the sections 404, 408 of the enclosure body and the overall safety device 400. These lower ribs 448a, 448b are also located, sized, and/or shaped to avoid interfering with the movement of the intra-osseous device through the safety device 400.

The upper ribs 456a, 456b of the second section 408, along with corresponding upper ribs of the first section 404, form an inner diameter like the lock feature 440. These upper ribs 456a, 456b also add rigidity to the sections 404, 408 but also guide part of the intra-osseous device along with the lock feature 440 and the parts of the sections 404, 408 that form the second aperture 428. More specifically, these features 440, 428, 456a, 456b form substantially circular inner diameters that are equal in some embodiments and that are approximately the same size as, for example, the barrel of an intra-osseous device to maintain the orientation of the intra-osseous device relative to the safety device 400. An example of an intra-osseous device 476 is shown in FIG. 6B where the intra-osseous device is a syringe with a barrel 480, a base 488 selectively connected to the barrel 480, a needle 496 connected to the base 488, and a plunger 500. The outer diameter 484 of the barrel 480 is approximately the same as, but still smaller than, the inner diameters established by the lock feature 440, the second aperture 428 and the upper ribs 456a, 456b. In addition, the outer diameter 492 of the base 488 is smaller than the diameter 424 of the first aperture 416 but larger than the inner diameters established by the lock feature 440, the second aperture 428 and the upper ribs 456a, 456b to hold the base 488 and the needle 496 within the safety device 400 as explained in further detail herein.

Referring again to FIG. 6A, a first guide tab 452a is positioned on the first section 404, and a second guide tab 452b is positioned on the second section 408 to retain part of the intra-osseous device within the safety device 400. Each tab 452a, 452b is oriented along a longitudinal axis or dimension of the safety device 400, and each tab 452a, 452b is connected to the sections 404, 408 at a lower end. This leaves the upper ends of the tabs 452a, 452b free-standing and deflectable to trap part of the intra-osseous device in a gap between the upper ends of the tabs 452a, 452b as described in further detail herein.

Referring to FIG. 6B, the intra-osseous device 476 is positioned in the safety device 400 from a side or lateral direction rather than a longitudinal direction to reduce the likelihood of an accidental needle stick. Then, the sections 404, 408 of the safety device 400 can move from an open position as shown in FIGS. 6A and 6B to a closed position as shown in FIGS. 6C and 6D. To help hold the sections 404, 408 in the closed position, a protrusion 460 extends from the first section 404, and a recess 472 extends into the second section 408. The protrusion 460 has a ramp portion or surface 468 and a shoulder portion or surface 464. As the sections 404, 408 close, and the protrusion 460 extends into the recess 472, the ramp portion 468 contacts an edge of the second section 408 to deflect the protrusion 460 from an original position to a deflected position. As the protrusion 460 advances and the ramp portion 468 clears the edge, the protrusion 460 deflects back to the original position, and the shoulder portion 464 holds the protrusion 460 within the recess 472 and holds the sections 404, 408 in the closed position. As shown, the safety device 400 has two sets of protrusions 460 and recesses 472, but it will be appreciated that the safety device 400 can have greater or fewer than two sets of protrusions and recesses.

With reference to FIGS. 6C and 6D, the sections 404, 408 of the safety device 400 have moved from an open position to a closed position to hold the intra-osseous device 476 within the safety device 400. FIGS. 6C and 6D also show how the intra-osseous device 476 moves relative to the intra-osseous device 400 from a first position in FIG. 6C to a second position in FIG. 6D. In the first position, the needle of the intra-osseous device 476 can extend into an osseous material such as the bone of a patient to inject and or remove material. At this point, the lower surface of the safety device 400 may contact a skin surface of a patient to maintain the orientation of the needle and prevent the needle from damaging bone or soft tissue of the patient. Then, the intra-osseous device 476 can be retracted through the safety device 400 to the second position. The safety device 400 maintains the orientation of the safety device 400 during retraction to, again, prevent the needle from damaging bone or soft tissue. Once in the second position, embodiments of the present disclosure provide a lock feature and/or locking function that holds at least part of the intra-osseous device 476 in the second position with the needle safely within the safety device 400 to prevent an accidental needle stick. Subsequently, in some embodiments, the barrel and plunger of the intra-osseous device 476 can be removed from a base and needle such that the barrel and plunger can be reused while the base and needle are safely stored with the safety device 400. In addition, lines C-C and D-D are shown in FIGS. 6C and 6D, respectively.

The material used to form the safety device 400 can add further functionality to the safety device 400. In some embodiments, the safety device 400 is made from K-Resin KR01 that is a clear styrene butadiene block copolymer. One or more surfaces of the safety device 400 is finished according to the SPI-B1 standard. As a result, the safety device 400 is not opaque and at least some light passes through the safety device 400. Thus, a user can see when the intra-osseous device is secured within the safety device. This visual inspection further improves safety and reduces the likelihood of accidental needle sticks. It will be appreciated that the safety device 400 can be made from any material, including materials that are opaque or allow at least some like to pass through the safety device 400.

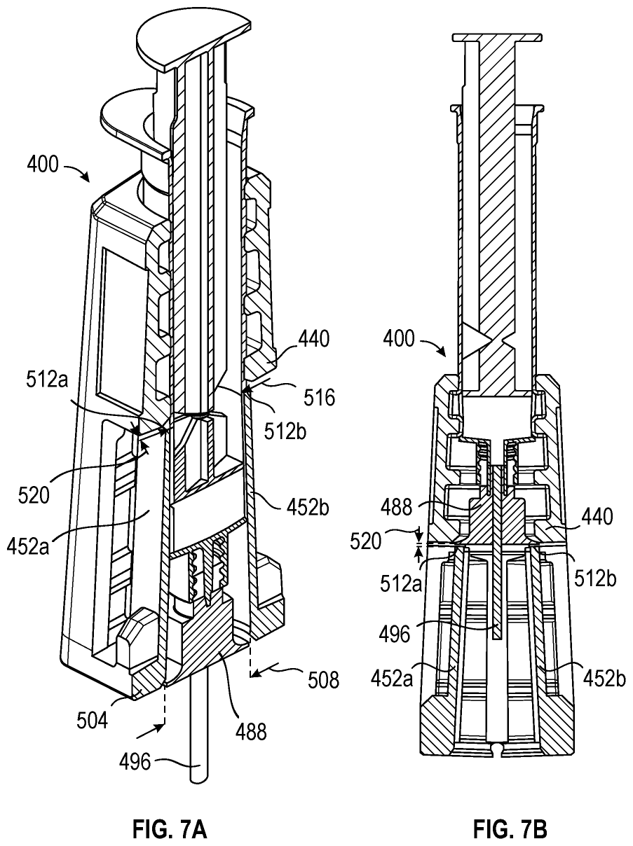

With reference to FIGS. 7A and 7B, cross-sectional views taken along lines C-C of FIG. 6C and D-D of FIG. 6D are provided, respectively. Specifically, these figures show the tabs 452a, 452b that guide a base 488 of the intra-osseous device from a first position to a second position relative to the safety device 400, and then retain the intra-osseous device within the safety device 400. As shown, the tabs 452a, 452b extend from a bottom portion 504 of enclosure body of the safety device 400, and in this embodiment, the tabs 452a, 452b have respective upper ends 512a, 512b that are free-standing and deflectable. FIG. 7A shows that the tabs 452a, 452b define an aperture with a lower diameter 508 that is the conterminous, and therefore, equal to the diameter of the first aperture. In other embodiments, it will be appreciated that this lower diameter 508 may be separate and distinct from the diameter of the first aperture. Next, the upper ends 512a, 512b define an upper diameter 516 that is smaller than the lower diameter 508. More specifically, the lower diameter 508 is larger than the outer diameter of the base 488, and the upper diameter 516 is smaller than the outer diameter of the base 488. Thus, as the intra-osseous device moves relative to the safety device 400, the base 488 of the intra-osseous device moves through the first aperture of the safety device 400, moves along the tabs 452a, 452a and eventually deflects the tabs 452a, 452b outward as the base 488 moves closer to the upper ends 512a, 512b of the tabs 452a, 452b. Once the base 488 clears the upper ends 512a, 512b, the tabs 452a, 452b deflect back inward to their original positions. At this point, the base 488 is in the position shown in FIG. 7B, and the base 488 is secured in a gap 520 between the upper ends 512a, 512b and the lock feature 440. Now the needle 496 is secured within the safety device 400 to prevent an accidental stick injury. Subsequently, the rest of the intra-osseous device can be removed from the base 488 and the needle 496.

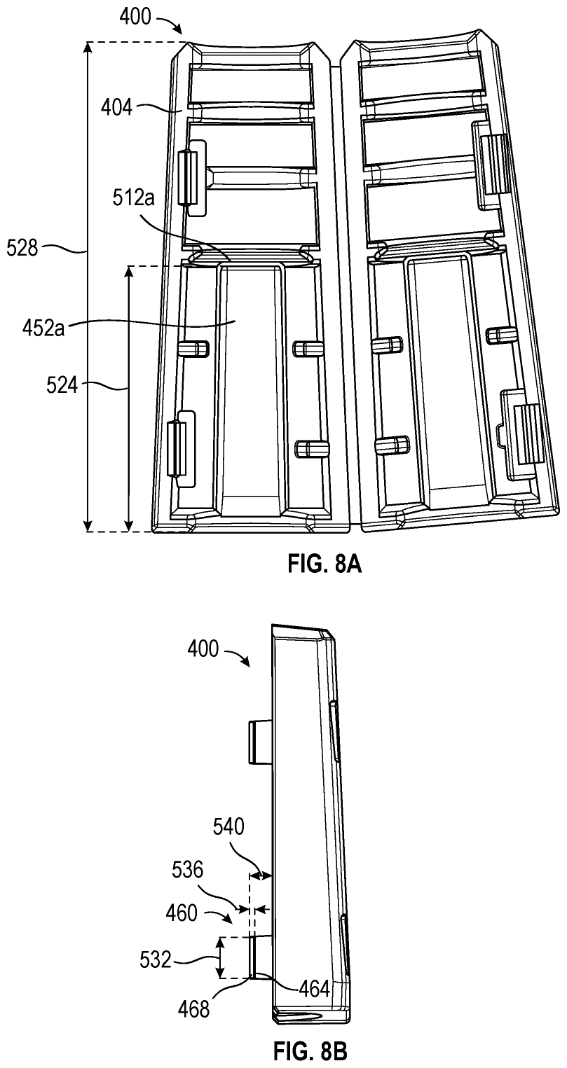

With reference to FIGS. 8A and 8B, a front elevation view and a side elevation view of the safety device 400 are provided, respectively. FIG. 8A shows various dimensions of the first section 404 of the safety device 400, which may also correspond to the second section as well. As described herein, the safety device 400 has a guide tab 452a with an upper end 512a to secure a base of an intra-osseous device within the safety device 400. The tab 452a has a height 524 that can be between approximately 1 and 3 inches in some embodiments. The term “approximately” may imply a variation of +/−% on a relative basis. In various embodiments, the height 524 is approximately 1.84+/−0.02 inches. The first section 404, and the overall enclosure body and safety device 400, also has a height 528, which can be between approximately 3 and 5 inches in some embodiments. In various embodiments, the height 528 is approximately 3.4 inches. These heights 524, 528 can also be described in relative terms. For example, the height 524 of the tab 452a is greater than half of the height 528 of the first section 404 such that an upper end 512a of the tab 452a is closer to the upper or second end of the safety device 400 rather than the lower or first end to provide space to receive and store the needle of an intra-osseous device.

FIG. 8B shows various dimensions of a protrusion 460 that secures the sections in a closed position. As described herein, the protrusion 460 has a shoulder portion 464 and a ramp portion 468. In some embodiments, the protrusion 460 has a height 532 that is between approximately 0.1 and 1 inch. In various embodiments, the height 532 is approximately 0.363 inches. The shoulder and ramp portions 464, 468 have a thickness 536 that is between approximately 0.02 and 0.1 inches in some embodiments. In various embodiments, the thickness 536 is approximately 0.05 inches. A distal edge of the protrusion 460 extends above the edge of the safety device 400 by a distance 540 that is between approximately 0.1 and 0.5 inches in some embodiments. In various embodiments, the distance 540 is approximately 0.2 inches.

The description of the present disclosure has been presented for purposes of illustration and description, but is not intended to be exhaustive or limiting of the disclosure to the form disclosed. Many modifications and variations will be apparent to those of ordinary skill in the art. The embodiments described and shown in the figures were chosen and described in order to best explain the principles of the disclosure, the practical application, and to enable those of ordinary skill in the art to understand the disclosure.

While various embodiments of the present disclosure have been described in detail, it is apparent that modifications and alterations of those embodiments will occur to those skilled in the art. Moreover, references made herein to “the invention” or aspects thereof should be understood to mean certain embodiments of the present disclosure and should not necessarily be construed as limiting all embodiments to a particular description. It is to be expressly understood that such modifications and alterations are within the scope and spirit of the present disclosure, as set forth in the following claims.

What is claimed is:

1. A safety device for an intra-osseous device, the safety device comprising:
an enclosure body having a first section and a second section, wherein the first and second sections are connected with a joint so that the first and second sections can move between an open position and a closed position, wherein the enclosure body holds the intra-osseous device in the closed position;
an internal cavity defined by the enclosure body when the first and second sections are in the closed position, wherein the internal cavity extends between a first aperture and a second aperture, and a diameter of the first aperture is larger than a diameter of the second aperture; and
a lock feature on interior surfaces of the first and second sections, wherein an inner diameter defined by the lock feature is less than the diameter of the first aperture to secure part of the intra-osseous device within the safety device.

2. The safety device of claim 1, further comprising:
a first tab on the interior surface of the first section;
a second tab on the interior surface of the second section, wherein the first and second tabs taper from the diameter of the first aperture to a smaller diameter at upper ends of the first and second tabs; and
a gap between the upper ends of the first and second tabs and the lock feature, wherein part of the intra-osseous device is adapted to deflect the first and second tabs outward until the part passes the first and second tabs, then the first and second tabs deflect inward, and the part is secured within the gap.

3. The safety device of claim 2, wherein the upper ends of the first and second tabs are closer to the second aperture at a second end of the enclosure body than the first aperture at a first end of the enclosure body.

4. The safety device of claim 2, wherein the smaller diameter defined by the upper ends of the first and second tabs is less than the inner diameter defined by the lock feature.

5. The safety device of claim 2, wherein the first tab is connected to the first section at only a lower end of the first tab, and the second tab is connected to the second section at only a lower end of the second tab.

6. The safety device of claim 1, further comprising:
a protrusion extending from the first section; and
a recess extending into the second section, wherein the protrusion extends into the recess to secure the first and second sections in the closed position.

7. The safety device of claim 6, wherein the protrusion has a ramp portion and a shoulder portion, wherein the ramp portion contacts an edge of the second section to defect the protrusion as the protrusion extends into the recess, and the shoulder portion retains the protrusion within the recess and secures the first and second sections in the closed position.

8. The safety device of claim 1, further comprising:
at least one support rib of the first and second sections that is positioned between the lock feature and the second aperture, wherein the at least one support rib defines an aperture with a diameter that is equal to the diameter of the second aperture.

9. The safety device of claim 1, wherein the enclosure body has an exterior surface defined by the first and second sections, and the exterior surface tapers from a first diameter at a first end of the enclosure body to a smaller second diameter at a second end of the enclosure body.

10. The safety device of claim 1, wherein the enclosure body is made from a material that allows at least some light to pass through the enclosure body.

11. A method of using a safety device with an intra-osseous device, comprising:
providing the safety device that has:
an enclosure body with a first section and a second section that are moveable between an open position and a closed position;
a lock feature on interior surfaces of the first and second sections;
positioning the intra-osseous device within the enclosure body and moving the first and second sections from the open position to the closed position to hold the intra-osseous device within the enclosure body; and
moving the intra-osseous device through the enclosure body along a longitudinal axis of the enclosure body until the lock feature secures a base portion of the intra-osseous device, and a needle of the intra-osseous device is safety positioned within the enclosure body.

12. The method of claim 11, further comprising:
deflecting a first tab on the first section and deflecting a second tab on the second section as the intra-osseous device is moved through an internal cavity of the enclosure body until the base portion passes upper ends of the first and second tabs, then the first and second tabs deflect back inward, and the base portion is secured between the upper ends of the first and second tabs and the lock feature.

13. The method of claim 11, further comprising:
inserting a protrusion of the first section into a recess of the second section to secure the first and second sections in the closed position.

14. The method of claim 13, further comprising:
contacting a ramp portion of the protrusion against an edge of the second section as the protrusion is inserted into the recess to deflect the protrusion from an original position to a deflected position; and
moving the ramp portion past the edge such that the protrusion reverts to the original position, and a shoulder of the protrusion retains the protrusion within the recess and secures the first and second sections in the closed position.

15. The method of claim 11, further comprising:
providing the safety device that has:
an internal cavity extending between a first aperture at a first end of the enclosure body and a second aperture at a second end of the enclosure body, wherein diameter of the first aperture is larger than a diameter of the second aperture such that the base portion can pass through the first aperture but not the second aperture.

16. The method of claim 11, further comprising:
removing the base portion of the intra-osseous device from a barrel of the intra-osseous device to leave the base portion and the needle of the intra-osseous device safely positioned within the enclosure body.

17. The method of claim 11, further comprising:
extending the intra-osseous device into an osseous material as the intra-osseous is held within the safety device; and
extending a plunger into a barrel of the intra-osseous device to deposit a fluid stored within the barrel to the osseous material.

18. The method of claim 11, further comprising:
extending the intra-osseous device into an osseous material as the intra-osseous is held within the safety device; and
retracting a plunger from a barrel of the intra-osseous device to remove part of the osseous material.

19. The method of claim 11, further comprising:
guiding the intra-osseous device along the longitudinal axis with a first support rib extending from the first section and a second support rib extending from the second section, wherein the first and second support ribs define an inner diameter that is equal to an inner diameter defined by the lock feature.

20. A safety device for an intra-osseous device, the safety device comprising:
an enclosure body having a first section and a second section, wherein the first and second sections are connected with a joint so that the first and second sections can move between an open position and a closed position, wherein the enclosure body holds the intra-osseous device in the closed position;
a protrusion extending from the first section;
a recess extending into the second section, wherein the protrusion extends into the recess to secure the first and second sections in the closed position;
an internal cavity defined by the enclosure body when the first and second sections are in the closed position, wherein the internal cavity extends between a first aperture and a second aperture, and a diameter of the first aperture is larger than a diameter of the second aperture;
a lock feature on interior surfaces of the first and second sections, wherein an inner diameter defined by the lock feature is less than the diameter of the first aperture;
a first tab on the interior surface of the first section;
a second tab on the interior surface of the second section, wherein the first and second tabs taper from the diameter at the first aperture to a smaller diameter defined by upper ends of the first and second tabs; and
a gap between the upper ends of the first and second tabs and the lock feature, wherein part of the intra-osseous device has a diameter that is larger than the diameter of the first aperture but less than the inner diameter defined by the lock feature such that the part of the intra-osseous device is adapted to deflect the first and second tabs outward until the part passes the first and second tabs, then the first and second tabs deflect inward, and the part is secured within the gap.
